(19) United States
(12) Patent Application Publication
(10) Pub. No.: US 2018/0249968 A1
(43) Pub. Date: Sep. 6, 2018

(54) INFORMATION PROCESSING DEVICE, CONTROL METHOD FOR INFORMATION PROCESSING DEVICE, AND STORAGE MEDIUM

(71) Applicant: NEC Corporation, Tokyo (JP)

(72) Inventors: Tatsushi YASUDA, Tokyo (JP); Masato KITADA, Tokyo (JP)

(73) Assignee: NEC Corporation, Tokyo (JP)

(21) Appl. No.: 15/760,050

(22) PCT Filed: Jun. 3, 2016
(86) PCT No.: PCT/JP2016/066670

§ 371 Date: Mar. 14, 2018

(30) Foreign Application Priority Data
Sep. 24, 2015 (JP) 2015-186278

(51) Int. Cl. A61B 5/00; A61B 5/0205; A63B 24/00; G16H 50/20

(52) U.S. Cl. CPC A61B 5/7292; A61B 5/02055; A61B 5/02438; A63B 24/0062; G16H 50/20; A61B 5/681

(57) ABSTRACT

This information processing device selects a sensor to be controlled according to the activity plan of a user. The information processing device is attached to the user's body. The information processing device is provided with: a plurality of sensors; an acquisition means for acquiring an activity plan of the user; and a control means that selects a sensor to be controlled from among the plurality of sensors on the basis of the acquired activity plan. The control means activates the sensor suited for the activity plan from among the plurality of sensors and inactivates sensors that are not suited for the activity plan from among the plurality of sensors.

### TECHNICAL FIELD

The present invention relates to an information processing device, a control method for the information processing device, and a control program for the information processing device.

### BACKGROUND ART

In the technical field described above, PTL 1 discloses a technique that determines, by using a second timer, whether or not a measurement has been executed at a preset timing by using a first timer. PTL 2 discloses a technique that determines an optimum combination of motion measurement devices for identifying an action of a person depending on a location of the subject and identifies an action of the person, based on measured values from the determined motion measurement devices. PTL 3 discloses a technique in which, when a first sensor out of two sensors of the same type is operating, a second sensor is deactivated and, when the second sensor is operating, the first sensor is deactivated. PTL 4 discloses a technique in which power management depending on a result of actual measurement of biological information is performed. PTL 5 discloses a technique in which, in order to measure biological information required for each individual user during a required period of time, a sensor is activated and a measurement is executed for a predetermined period of time in a particular state.

### CITATION LIST

### Patent Literature

[PTL 1] International Publication No. WO 2014/057625

### [PTL 2] Japanese Laid-open Patent Publication No. 2009-134590

[PTL 3] International Publication No. WO 2012/079052

### [PTL 4] Japanese Laid-open Patent Publication No. 2015-123300

### [PTL 5] Japanese Laid-open Patent Publication No. 2015-112209

### SUMMARY OF INVENTION

### Technical Problem

However, none of the techniques described in the literatures cited above is capable of selecting a sensor to be controlled from among a plurality of sensors, based on an action plan of a user.

An object of the present invention is to provide a technique that solves the problem described above.

### Solution to Problem

To achieve the above object, an information processing device configured to be attached to a body of a user. The device includes:

a plurality of sensors;

an acquisition means that acquires an action plan of the user; and

a control means that selects a sensor to be controlled among the plurality of sensors, based on the acquired action plan.

To achieve the above object, a control method for an information processing device that includes a plurality of sensors and is configured to be attached to a body of a user. The control method includes:

an acquisition step of acquiring an action plan of the user; and

a control step of selecting a sensor to be controlled among the plurality of sensors, based on the acquired action plan.

To achieve the above object, a control program for an information processing device includes a plurality of sensors and is configured to be attached to a body of a user. The control program causes a computer to execute:

an acquisition step of acquiring an action plan of the user; and

a control step of selecting a sensor to be controlled among the plurality of sensors, based on the acquired action plan.

### Advantageous Effects of Invention

According to the present invention, a sensor to be controlled can be selected from among a plurality of sensors, based on an action plan of a user.

### BRIEF DESCRIPTION OF DRAWINGS

FIG. 1 is a block diagram illustrating a configuration of an information processing device according to a first example embodiment of the present invention.

FIG. 2 is a diagram illustrating an overview of an operation of an information processing device according to a second example embodiment of the present invention.

FIG. 3 is a block diagram illustrating a configuration of the information processing device according to the second example embodiment of the present invention.

FIG. 4A is a diagram illustrating one example of a sensor combination table provided in the information processing device according to the second example embodiment of the present invention.

FIG. 4B is a diagram illustrating one example of a sensor combination tale provided in the information processing device according to the second example embodiment of the present invention.

FIG. 4C is a diagram illustrating one example of a sensor activation condition table provided in the information processing device according to the second example embodiment of the present invention.

FIG. 5 is a block diagram illustrating a hardware configuration of the information processing device according to the second example embodiment of the present invention.

FIG. 6 is a flowchart illustrating a procedure of processing performed by the information processing device according to the second example embodiment of the present invention.

FIG. 7 is a block diagram illustrating a configuration of an information processing device according to a third example embodiment of the present invention.

FIG. 8 is a diagram illustrating one example of a user information table provided in the information processing device according to the third example embodiment of the present invention.

FIG. 9 is a block diagram illustrating a hardware configuration of the information processing device according to the third example embodiment of the present invention.

FIG. 10A is a flowchart illustrating a procedure of processing performed by the information processing device according to the third example embodiment of the present invention

FIG. 10B is another example of a flowchart illustrating a procedure of processing performed by the information processing device according to the third example embodiment of the present invention.

FIG. 11 is a block diagram illustrating a configuration of an information processing device according to a fourth example embodiment of the present invention.

FIG. 12 is a diagram illustrating one example of a related/associated mode table provided in the information processing device according to the fourth example embodiment of the present invention.

FIG. 13 is a block diagram illustrating a hardware configuration of the information processing device according to the fourth example embodiment of the present invention.

FIG. 14 is a flowchart illustrating a procedure of processing performed by the information processing device according to the fourth example embodiment of the present invention.

### DESCRIPTION OF EMBODIMENTS

Example embodiments for carrying out the present invention will be described below in detail by way of illustration with reference to drawings. However, configurations, numerical values, processing flows, functional elements and the like are illustrative only and are not intended to limit the technical scope of the present invention to the following description, and variations and modifications thereof may be arbitrarily made. Note that the term “action plan of a user” as used herein encompasses all activities that use sensors provided in an information processing device.

### First Example Embodiment

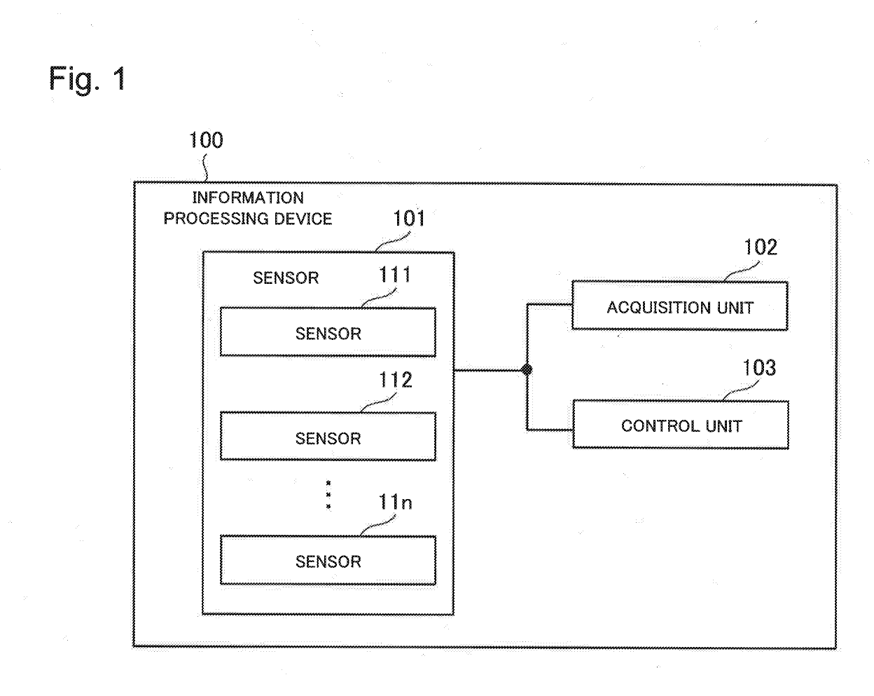

An information processing device 100 as a first example embodiment of the present invention will be described using FIG. 1. The information processing device 100 is a device that is intended to be attached to the body of a user and selects a sensor to be controlled in accordance with an action plan of the user.

As illustrated in FIG. 1, the information processing device 100 includes sensors 101, an acquisition unit 102, and a control unit 103. The sensors 101 include a plurality of sensors 111 to 11n. The acquisition unit 102 acquires an action plan of a user. The control unit 103 selects a sensor to be controlled from among the plurality of sensors 111 to 11n based on the acquired action plan.

According to the present example embodiment, a sensor to be controlled can be selected from among a plurality of sensors based on an action plan of a user.

### Second Example Embodiment

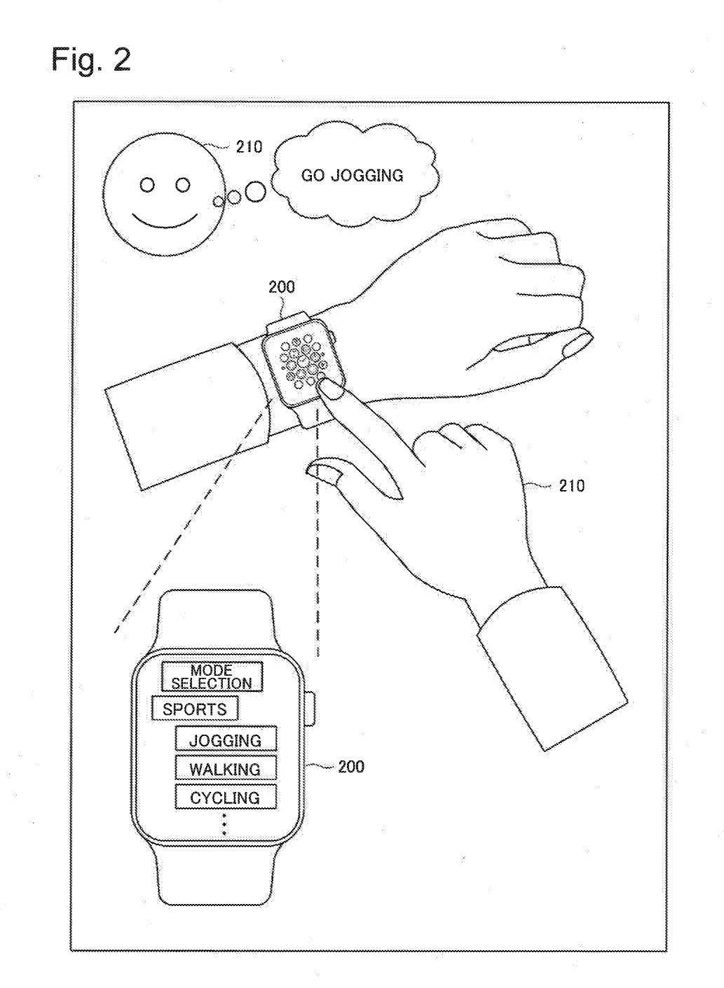

An information processing device according to a second example embodiment of the present invention will be described next using FIGS. 2 to 6. FIG. 2 is a diagram illustrating an overview of an operation of an information processing device 200 according to the present example embodiment. Note that while the information processing device 200 will be described below by taking a wristwatch-type wearable terminal as an example, wearable terminals that are applicable as the information processing device 200 are not limited to this. The information processing device 200 may be a wearable terminal of a type such as a spectacle-type, shoe-type, and clothing-type.

Assume for example that a user 210 wearing an information processing device 200 such as a wearable device round his/her left wrist is going to go jogging. Then the user 210 wants to measure and record conditions of the user 210 doing the exercise by using various sensors provided in the wearable device and functions of the wearable device. In such a case, it is very troublesome for the user 210 to find out which of the sensor is to be activated in accordance with the exercise or the activity that the user 210 is going to perform and make a setting to turn on or off each individual sensor. In the case of jogging, the user 210 has to perform operations to turn on, for example, a heart rate sensor, an acceleration sensor, and a global positioning system (GPS) sensor among the various sensors and operations to turn off the other sensors.

To address this, in the present example embodiment, once a user 210 inputs, sets or otherwise chooses an action plan of the user 210, the user 210 does not need to perform an operation to set each individual sensor and the information processing device 200 selects sensors to be controlled in accordance with the acquired action plan.

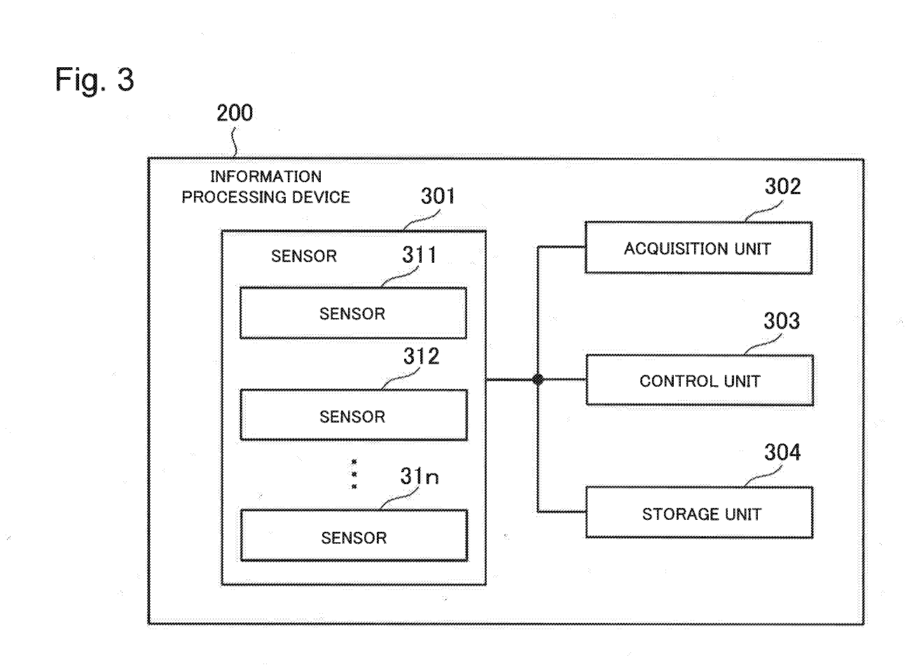

FIG. 3 is a block diagram illustrating a configuration of the information processing device 200 according to the present example embodiment. The information processing device 200 includes sensors 301, an acquisition unit 302, a control unit 303 and a storage unit 304. The sensors 301 include sensors 311 to 31n.

The sensors 301 are a set of a plurality of sensors 311 to 31n, which may be sensors of different types that detect different phenomena or may include several sensors of the same type that detect the same phenomenon. For example, the sensors 311 to 31n may be a heart rate sensor, an acceleration sensor, a GPS sensor, an ambient temperature sensor, a body temperature sensor, an altitude sensor, a humidity sensor, a lactate level sensor, a respiratory rate sensor, a blood flow sensor, an oxygen level sensor, a blood-sugar-level sensor, a perspiration sensor, an alcohol sensor and the like. However, the sensors 311 to 31n are not limited to these sensors and may be any sensors that are capable of measuring some physical quantities.

The acquisition unit 302 acquires an action plan of the user 210. Action plans of the user 210 include, for example, sports, healthcare, disease prevention and the like. These action plans are displayed on a screen for selecting an action plan of the user 210 or the like, as modes such as a sports mode, a healthcare mode, and a disease prevention mode, for example. Action plans included in the sports mode include, but not limited to, action plans such as jogging, walking, cycling, golf, mountain climbing, tennis, soccer, baseball, swimming, indoor jogging, indoor walking, and indoor cycling. Action plans included in healthcare mode include action plans such as fatigue level check and water intake check, for example. Action plans included in the disease prevention mode include action plans such as heat attack, high-altitude disease, diabetes, heart failure, and kidney disease, for example. Action plans included in the healthcare mode and disease prevention mode are not limited to these action plans.

The control unit 303 selects a sensor 301 to be controlled from among the plurality of sensors 311 to 31n based on an action plan of the user 210 acquired by the acquisition unit 302. For example, when the user 210 selects jogging in the sports mode as an action plan, the control unit 303 selects and activates the heart rate sensor, the acceleration sensor and the GPS sensor as sensors to be controlled. Thus, the control unit 303 includes the aspect of selecting and activating sensors 301 to be controlled and, conversely, includes the aspect of turning off sensors 301 that are not to be controlled. In other words, the control unit 303 activates sensors suitable for an action plan among the plurality of sensors 311 to 31n and, conversely, deactivates sensors that are not suitable for the action plan.

The control unit 303 also controls the sensitivity, precision, resolution, detection intervals, detection frequency and the like of the sensors 301. For example, when the user 210 is performing an exercise such as jogging at a constant speed, the control unit 303 lengthens the intervals of detection of acceleration by the acceleration sensor or decreases the frequency of detection by the acceleration sensor.

Further, when the user 210 wearing the information processing device 200 selects the jogging mode, for example, and it is raining at the start of the jogging or it starts raining during the jogging, it is detected that the user 210 is heavily sweating, due to perspiration and the rainwater. To address this, sensor control unit 303 may lower the sensitivity of the perspiration sensor or stop detection by the perspiration sensor.

Further, the number of sensors 311 to 31n, such as the perspiration sensor, attached to the body of the user 210 is not limited to one but a plurality of sensors may be attached to the body of the user 210. For example, when a plurality of perspiration sensors are attached to the body, the amounts of perspiration at different parts of the body can be measured.

The storage unit 304 stores action plans of the user 210 and sensors 301 to be controlled in association with each other.

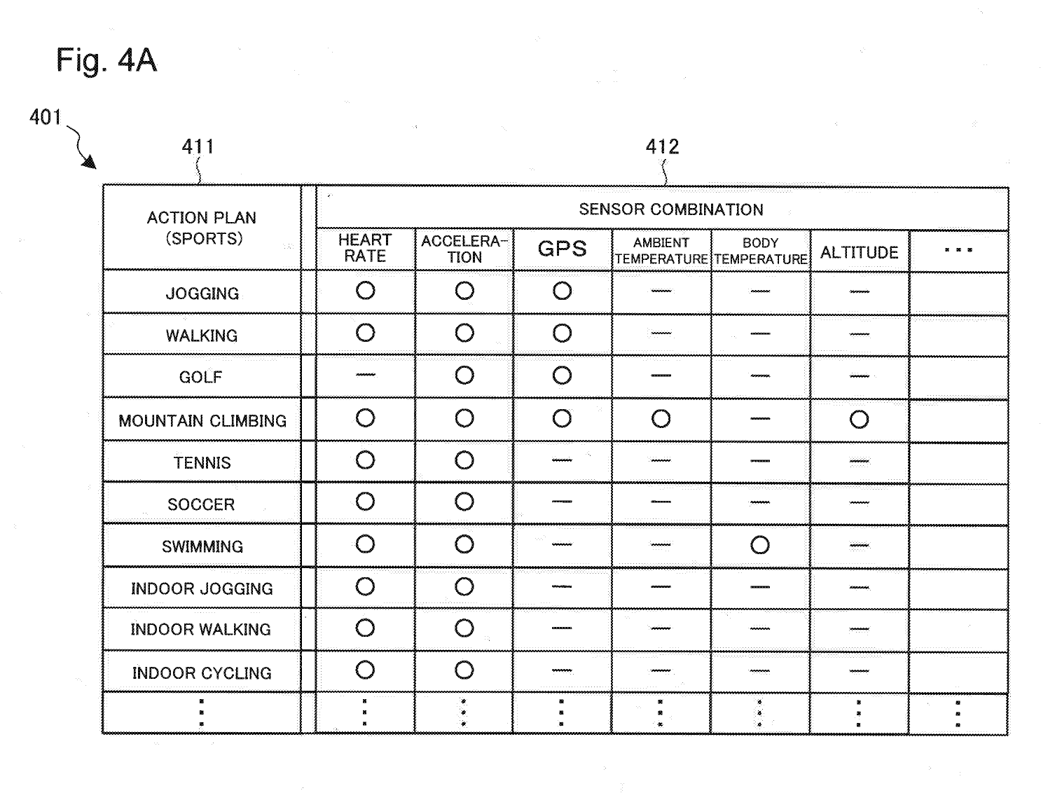

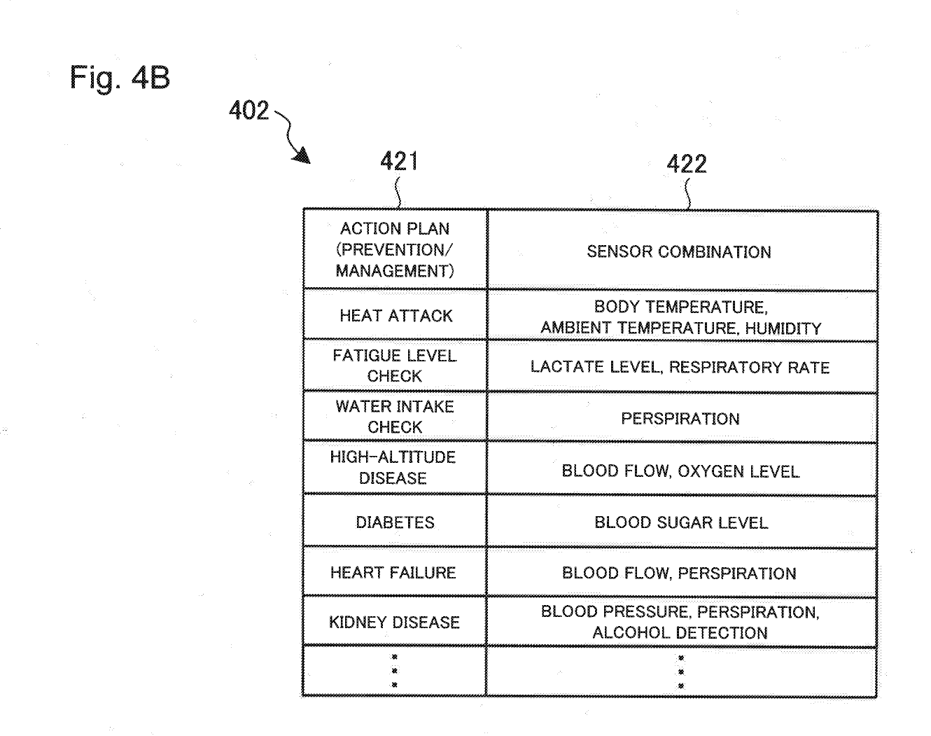

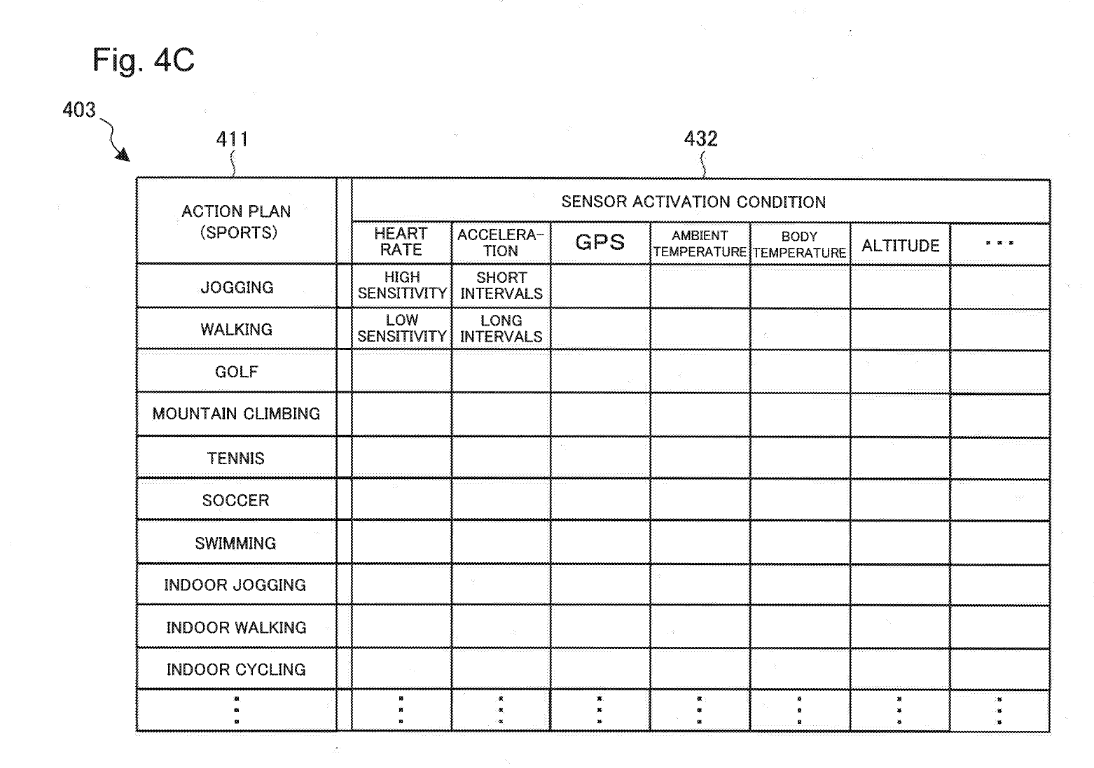

FIG. 4A is a diagram illustrating one example of a sensor combination table 401 provided in the information processing device 200 according to the present example embodiment. The sensor combination table 401 stores sensor combinations 412 in association with action plans (sports) 411.

The sensor combinations 412 are combinations of sensors 301 that are related to action plans (sports) 411, i.e. examples of combinations of sensors to be controlled. For example, when an action plan (sports) 411 is jogging, a combination of sensors 301 is the combination of three sensors: a heart rate sensor, an acceleration sensor, and a GPS sensor, which is a combination of sensors to be controlled. When an action plan (sports) 411 is golf, a combination of sensors 301 is the combination of the acceleration sensor and the GPS sensor; when an action plan (sports) 411 is mountain climbing, a combination of sensors is the combination of the heart rate sensor, the acceleration sensor, the GPS sensor, an ambient temperature sensor and an altitude sensor, which is a combination of sensors to be controlled.

The control unit 303 refers to the sensor combination table 401 and selects and controls a combination of sensors 301 that are related to an action plan. The method by which the control unit 303 selects a combination of sensors 301 is not limited to this. The control unit 303 may select and control sensors 301 to be used for an action plan acquired by the information processing device 200 that are suitable for the action plan on a case-by-case basis.

FIG. 4B is a diagram illustrating one example of a sensor combination table 402 provided in the information processing device 200 according to the present example embodiment. The sensor combination table 402 stores sensor combinations 422 in association with action plans (prevention/management) 421.

The sensor combinations 422 are combinations of sensors 301 related to action plans (prevention/management) 421, i.e. examples of sensors to be controlled. For example, when an action plan (prevention/management) 421 is heat attack, a combination of sensors 301 to be controlled is a combination of three sensors: a body temperature sensor, an ambient temperature sensor and a humidity sensor. When an action plan (prevention/management) 421 is diabetes, a sensor 301 to be controlled is a blood-sugar-level sensor. The blood-sugar-level sensor measures a blood sugar level of the user 210 by irradiating an arm of the user 210 with light, for example. Likewise, the lactate level sensor and the blood flow sensor measure a lactate level and a blood flow, respectively, by irradiating an arm of the user 210 with light. The selection unit 303 selects a combination of sensors 301 with reference to the sensor combination table 402.

FIG. 4C is a diagram illustrating one example of a sensor activation condition table 403 provided in the information processing device 200 according to the present example embodiment. The sensor activation condition table 403 stores sensor activation conditions 432 in association with action plans (sports) 411.

For example, as illustrated in FIG. 4A, sensors 301 to be controlled are the same for jogging and walking; similarly, sensors 301 to be controlled are the same for tennis and soccer. However, conditions for activating the sensors 301 are different for jogging and walking and for tennis and soccer. For example, in the case of jogging, it is preferable to set a higher sensor sensitivity for the heart rate sensor and shorter detection intervals for the acceleration sensor since jogging is a relatively heavy exercise. On the other hand, in the case of walking, it is preferable to set a lower sensor sensitivity for the heart rate sensor and longer detection intervals for the acceleration sensor since walking is a relatively light exercise. The control unit 303 refers to the sensor activation condition table 403 and changes conditions for activating sensors, such as the sensor sensitivities and detection intervals of sensors 301 to be controlled, as appropriate in accordance with an action plan.

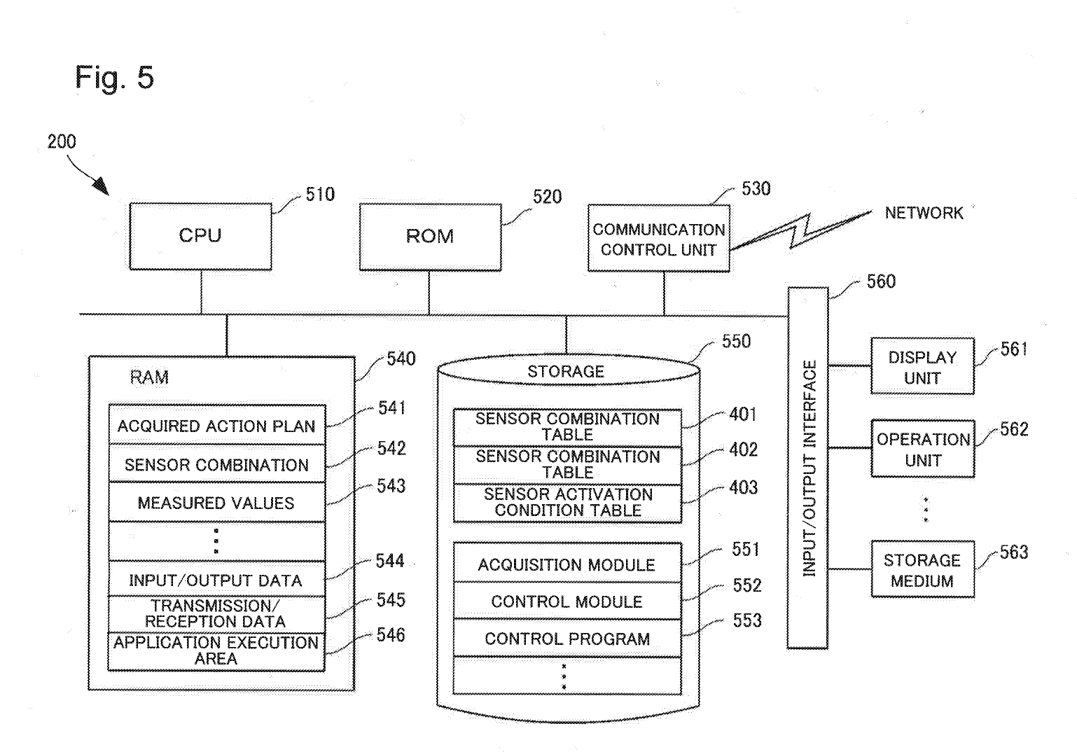

FIG. 5 is a block diagram illustrating a hardware configuration of the information processing device 200 according to the present example embodiment. A central processing unit (CPU) 510 is a processor for computation and control and executes a program to implement the functional components of the information processing device 200 in FIG. 3. A read only memory (ROM) 520 stores initial data, fixed data for programs and the like, and programs. A communication control unit 530 communicates with external devices via a network. Note that the CPU 510 is not limited to a single CPU but may be a plurality of CPUs or may include a graphics processing unit (GPU) for image processing. Desirably, the communication control unit 530 has a CPU independent of the CPU 510 and writes or reads transmission/reception data in an area in a random access memory (RAM) 504. Further, desirably, a direct memory access controller (DMAC) that transfers data between the RAM 540 and a storage 550 is provided (not depicted). In addition, desirably, an input/output interface 560 has a CPU independent of the CPU 510 and writes or reads input/output data in an area in the RAM 540. The CPU 510 therefore recognizes that data has been received or transferred in the RAM 540 and processes the data. Further, the CPU 510 provides a result of processing in the RAM 540 and allows the communication control unit 530, the DMAC, or the input/output interface 560 to transmit or transfer the result.

The RAM 540 is a random access memory used by the CPU 510 as a temporary-storage work area. An area for storing data required for implementing the present example embodiment is reserved in the RAM 540. An acquired action plan 541 is an action plan of the user 210 acquired by the information processing device 200. A sensor combination 542 is a combination of sensors 301 selected as sensors to be controlled, based on an acquired action plan. Measured values 543 are values measured by individual sensors 301. Input/output data 544 is data input and output via the input/output interface 560. Transmission/reception data 545 is data transmitted and received via the communication control unit 530. An application execution area 546 is an area used by an application in processing other than storage control.

The storage 550 stores a database and various parameters, or data or programs which will be described below, required for implementing the present example embodiment. Sensor combination tables 401, 402 and a sensor activation condition table 403 are tables configured as illustrated in FIGS. 4A to 4C.

The storage 550 further stores an acquisition module 551 and a control module 552. The acquisition module 551 is a module that acquires an action plan of the user 210. The control module 552 is a module that selects and controls sensors 301 to be controlled from among a plurality of sensors 301 based on an income↓ action plan. The modules 551 and 552 are loaded by the CPU 510 into the application execution area 546 of the RAM 540 and executed in the application execution area 546. A control program 553 is a program for controlling the entire information processing device 200.

The input/output interface 560 interfaces input/output data with input/output devices. A display unit 561 and an operation unit 562 are connected to the input/output interface 560. A storage medium 563 may also be connected to the input/output interface 560. In addition, a speaker, which is an audio output unit, a microphone, which is an audio input unit, or a GPS positioning unit may be connected to the input/output interface 560. Note that programs and data relating to general-purpose functions and other implementable functions of the information processing device 200 are not depicted in the RAM 540 and the storage 550 illustrated in FIG. 5.

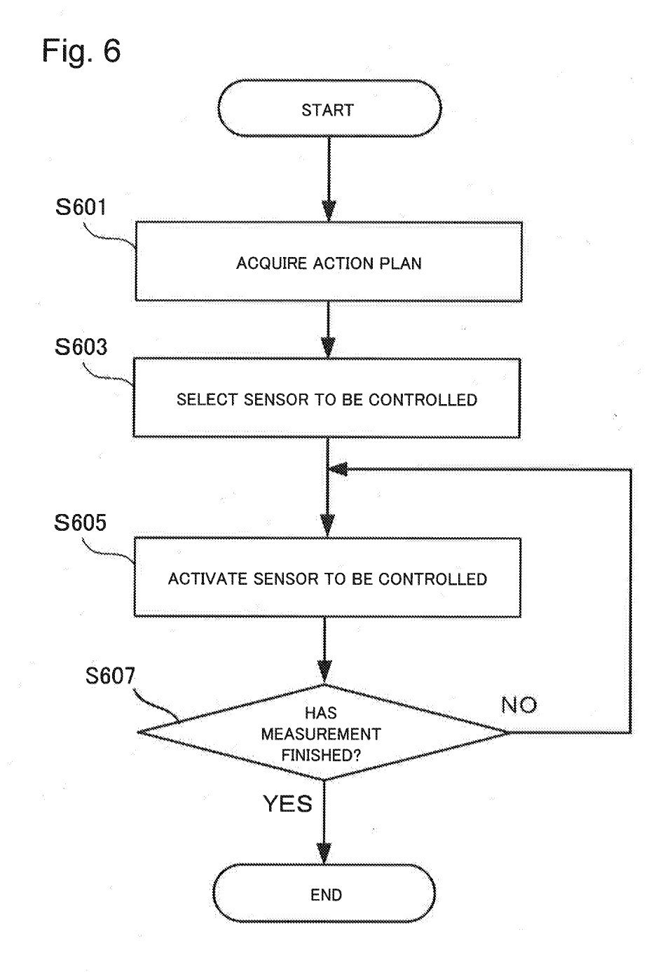

FIG. 6A is a flowchart illustrating a procedure of processing performed by the information processing device 200 according to the present example embodiment. The flowchart is executed by the CPU 510 in FIG. 5 by using the RAM 540 and implements the functional components of the information processing device 200 in FIG. 3.

In step S601, the acquisition unit 302 acquires an action plan of the user 210. In step S603, the control unit 303 selects a sensor to be controlled from among the plurality of sensors 301 based on the acquired action plan. In step S605, the control unit 303 activates the sensor to be controlled and makes a measurement. In step S607, the information processing device 200 determines whether to end the measurement. When the information processing device 200 determines not to end the measurement (NO in step S607), the information processing device 200 repeats step S605 and the subsequent step. When the information processing device 200 determines to end the measurement (YES in step S607), the information processing device 200 ends the processing. The determination as to whether to end the measurement may be based on, but not limited to, whether or not the user 210 has performed an operation to end the measurement, for example.

According to the present example embodiment, a sensor to be controlled among the plurality of sensors can be controlled, based on an action plan of a user. Further, since a sensor to be controlled is selected and activated from among a plurality of sensors in accordance with an action plan of a user, sensors can be automatically activated without the user having to find out sensors to be activated and perform on/off operations. Consequently, the user can know his/her performance and goal achievement level and the like of an exercise or an activity without having to make detailed settings of the information processing device.

### Third Example Embodiment

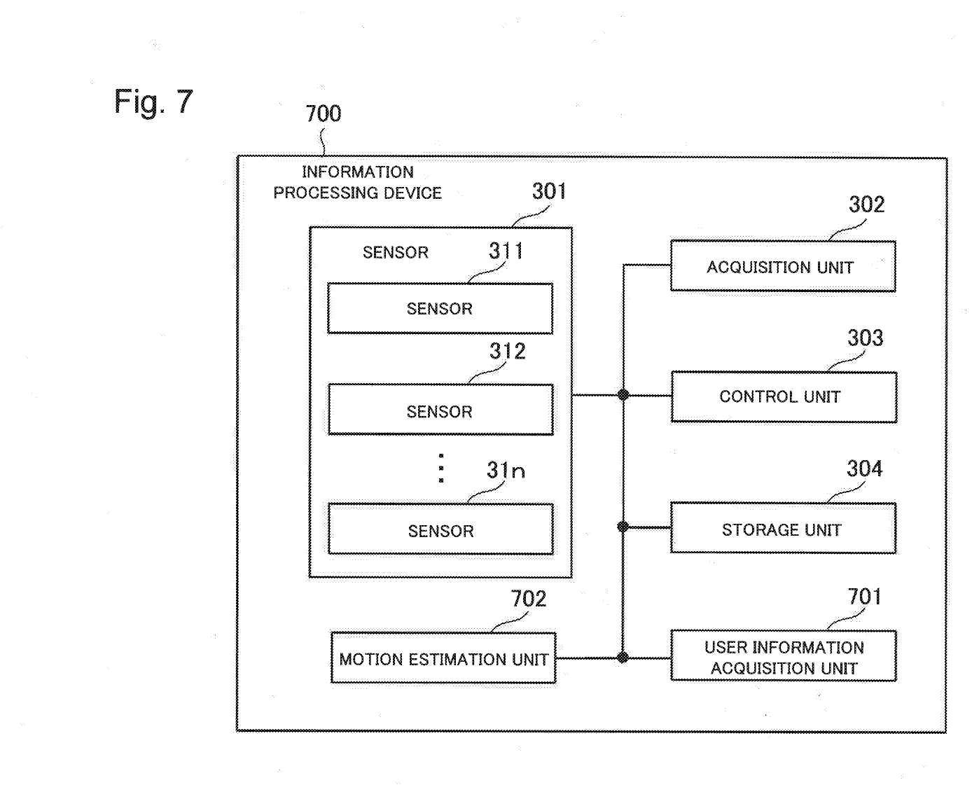

An information processing device according to a third example embodiment of the present invention will be described next using FIGS. 7 to 10. FIG. 7 is a block diagram for explaining a configuration of an information processing device 700 according to the present example embodiment. The information processing device 700 according to the present example embodiment differs from the second example embodiment described above in that the information processing device 700 includes a user information acquisition unit and a motion estimation unit. The rest of the configuration and operations are similar to the configuration and operations of the second example embodiment and therefore like configurations and operations will be given like reference numerals and detailed description thereof will be omitted.

The information processing device 700 includes a user information acquisition unit 701 and a motion estimation unit 702. The user information acquisition unit 701 acquires user information such as personal information and health conditions of a user. For example, when the user 210 is diabetic, a control unit 303 selects and activates a sensor 301 to be controlled, based on an action plan and user information acquired by the information processing device 200.

For example, the user 210 who is a diabetic and is measuring his/her blood sugar level at predetermined intervals or continuously by using the information processing device 700 sets jogging as an action plan. Then, the control unit 303 of the information processing device 700 selects and activates a heart rate sensor, an acceleration sensor and a GPS sensor since jogging is set. However, by doing this, a blood-sugar-level sensor is turned off and the diabetic user 210 cannot measure his/her blood sugar level at predetermined intervals or otherwise. The control unit 303 therefore selects and activates the blood-sugar-level sensor in addition to the heart rate sensor, the acceleration sensor and the GPS sensor as sensors 301 to be controlled by taking into consideration the user information acquired by the user information acquisition unit 701. Since the control unit 303 controls sensors in this way, the blood-sugar-level sensor is prevented from automatically turning off, thereby allowing the user 210 to measure his/her blood sugar level even during jogging.

Further, the control unit 303 may select and activate a sensor 301 to be controlled, based on a schedule of the user 210 that is contained in user information. For example, the user information acquisition unit 701 acquires a schedule of the user 210 from a scheduler installed in the information processing device 700 and automatically collects an action plan of the user from the acquired schedule. Based on the automatically collected action plan of the user 210, the control unit 303 selects and activates a sensor 301 to be controlled. For example, in the case where the user 210 habitually starts jogging at 7 a.m. every morning, once the user 210 registers information indicating that the user 210 starts jogging at 7 a.m. in the scheduler, then, at 7 a.m., controller 303 selects and activates sensors to be controlled during jogging. In this way, the information processing device 700 automatically activates sensors 301 at 7 a.m. every morning without the user 210 having to operate the information processing device 700 to set a jogging mode at 7 a.m. every morning.

The motion estimation unit 702 estimates a motion of the user 210 from measured values from active sensors 301. For example, when the acceleration sensor and the GPS sensor are in the on state and the user 210 starts to move, the motion estimation unit 702 estimates a motion that the user 210 may make from acceleration measured by the acceleration sensor, speed derived from the acceleration, or a length of move, travel time, and the like derived from the measured value from the GPS sensor. For example, when the user 210 has traveled a significant distance in a short time or the acceleration increases in a short time and the average movement rate increases, the motion estimation unit 702 can estimate that the user 210 has started jogging. Then the information processing device 700 estimates an action plan of the user 210 and automatically selects sensors 301 to be controlled without the user 210 having to set jogging as an action plan. In this way, the motion estimation unit 702 estimates a motion of the user 210 to allow the user 210 to measure his/her performance of an exercise when the user 210 feel like doing so, without having to set an action plan.

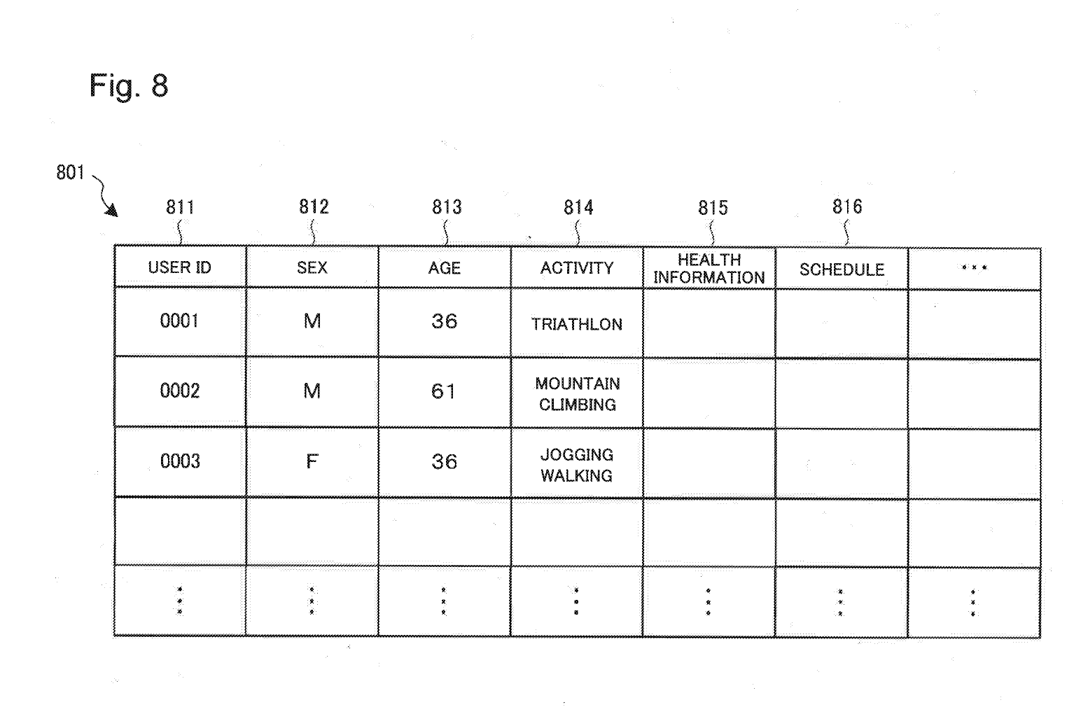

FIG. 8 is a diagram illustrating one example of a user information table 801 provided in the information processing device 700 according to the present example embodiment. The user information table 801 stores information such as the sex 812, the age 813, an activity 814, health information 815, and a schedule 816 in association with a user identifier (ID) 811. The user information acquisition unit 701 refers to the user information table 801 and acquires user information concerning the user 210 wearing the information processing device 700. Note that the method by which the user information acquisition unit 701 acquires user information is not limited to this. For example, the user information acquisition unit 701 may acquire user information from data manually input by the user 201.

The control unit 303 may determine, based on the user information acquired by the user information acquisition unit 701, what kind of exercise or activity the user 210 does and may select and activate sensors 301 to be controlled.

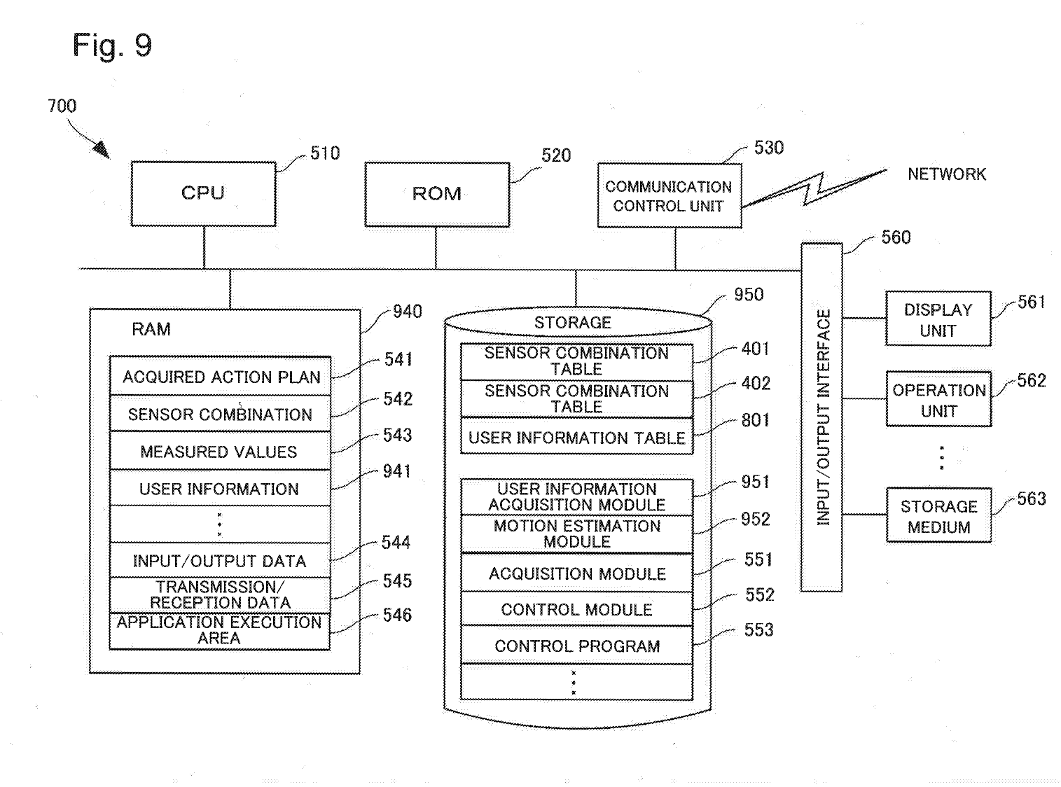

FIG. 9 is a block diagram illustrating a hardware configuration of the information processing device 700 according to the present example embodiment. A RAM 940 is a random access memory used by the CPU 510 as a temporary-storage work area. An area for storing data required for implementing the present example embodiment is reserved in the RAM 940. User information 941 is information such as personal information and health information concerning the user 210 wearing the information processing device 700 and is temporarily stored in the RAM 940. The user information 941 may be loaded from a database that stores user information, not depicted, into the RAM 940.

A storage 950 stores a database and various parameters, or data or programs, which will be described below, required for implementing the present example embodiment. A user information table 801 is a table configured as illustrated in FIG. 8 and is stored in the storage 950.

The storage 950 further stores a user information acquisition module 951 and a motion estimation module 952. The user information acquisition module 951 is a module that acquires user information. The motion estimation module 952 is a module that estimates a motion of the user 210. The modules 951 and 952 are loaded by the CPU 510 into an application execution area 546 of the RAM 940 and is executed in the application execution area 546.

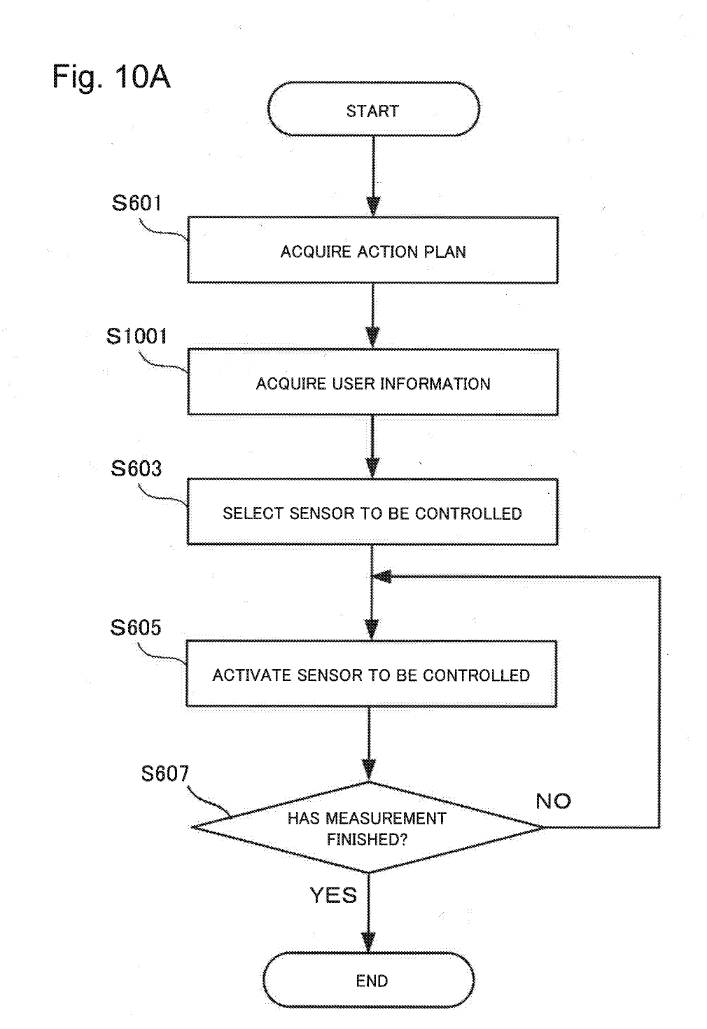

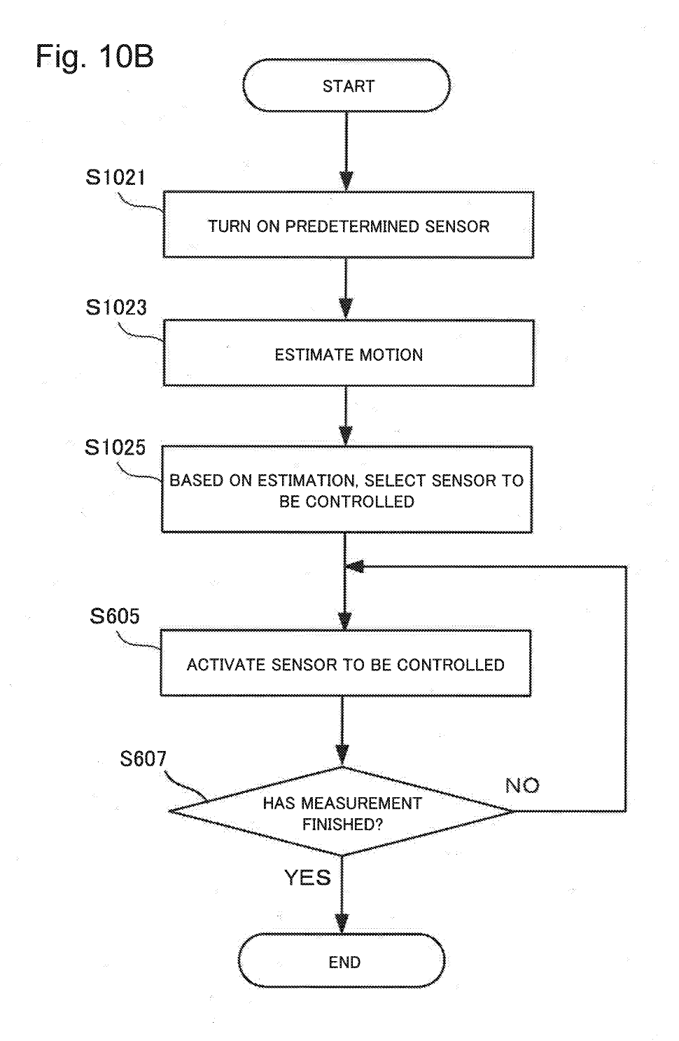

FIG. 10A is a flowchart illustrating a procedure of processing performed by the information processing device 700 according to the present example embodiment. The flowchart is executed by the CPU 510 in FIG. 9 by using the RAM 940 and implements the functional components of the information processing device 700 in FIG. 7. In step S1001, the user information acquisition unit 701 acquires user information concerning the user 210 wearing the information processing device 700.

FIG. 10B is another example of a flowchart illustrating a procedure of processing performed by the information processing device 700 according to the present example embodiment. The flowchart is executed by the CPU 510 in FIG. 9 by using the RAM 940 and implements the functional components of the information processing device 700 in FIG. 7.

In step S1021, the information processing device 700 turns on predetermined sensors, for example the acceleration sensor and the GPS sensor and the like. In step S1023, the motion estimation unit 702 estimates a motion of the user by using the sensors 301 that are turned on. In step S1025, based on the motion estimation, the control unit 303 selects and activates a sensor 301 to be controlled.

According to the present example embodiment, a combination of sensors to be activated can be selected from among a plurality of sensors in accordance with a purpose of use of the information processing device. Further, a sensor to be activated can be selected based on user information. Moreover, a combination of sensors to be activated can be selected by estimating a motion of the user. Accordingly, the user can measure and evaluate his/her performance and goal achievement level of an exercise and an activity without having to perform complicated setting operations of the information processing device.

### Fourth Example Embodiment

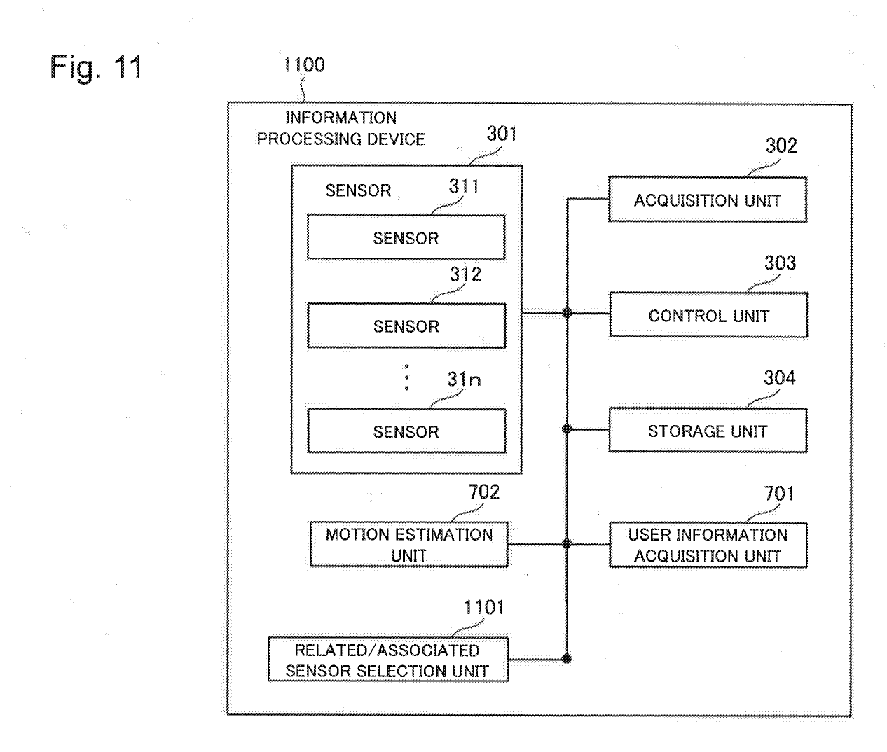

An information processing device according to a fourth example embodiment of the present invention will be described next using FIGS. 11 to 14. FIG. 11 is a block diagram for explaining a configuration of an information processing device 1100 according to the present example embodiment. The information processing device 1100 according to the present example embodiment differs from the third example embodiment described above in that the information processing device 1100 includes a related/associated sensor selection unit. The rest of the configuration and operations are similar to the configuration and operations of the third example embodiment and therefore like configurations and operations will be given like reference numerals and detailed description thereof will be omitted.

A related/associated sensor selection unit 1101 selects a sensor that is associated with a combination of sensors 301 selected by a selection unit 303. For example, when a user 210 sets jogging, associated or related sensors are a combination of sensors 301 capable of detecting heat attack or a combination of sensors 301 capable of checking a fatigue level.

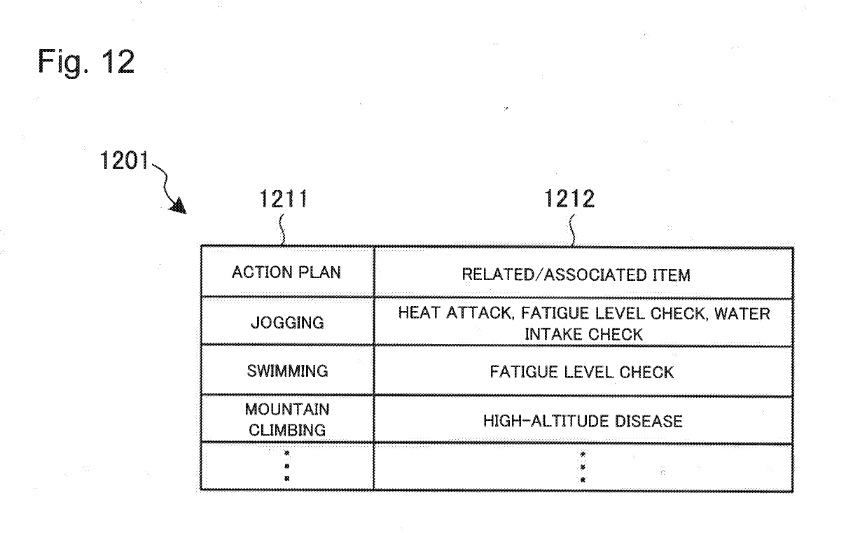

FIG. 12 is a diagram illustrating one example configuration of a related/associated item table 1201 provided in the information processing device 1100 according to the present example embodiment. The related/associated item table 1201 stores related/associated items 1212 in association with purposes 1211. A related/associated item 1212 is not a main sensor or a main measurement item for a purpose 1211 but is an item about which the user 210 needs to be careful in an exercise or activity and is an item related to or associated with the main purpose. For example, in the case where the purpose 1211 is jogging, the related/associated items 1212 are heat attack, fatigue level check, water intake check and the like. The related/associated sensor selection unit 1101 refers to the related/associated item table 1201 to determine a related/associated item 1212 for a main purpose and selects a combination of sensors 301 to be used for the determined related/associated item 1212.

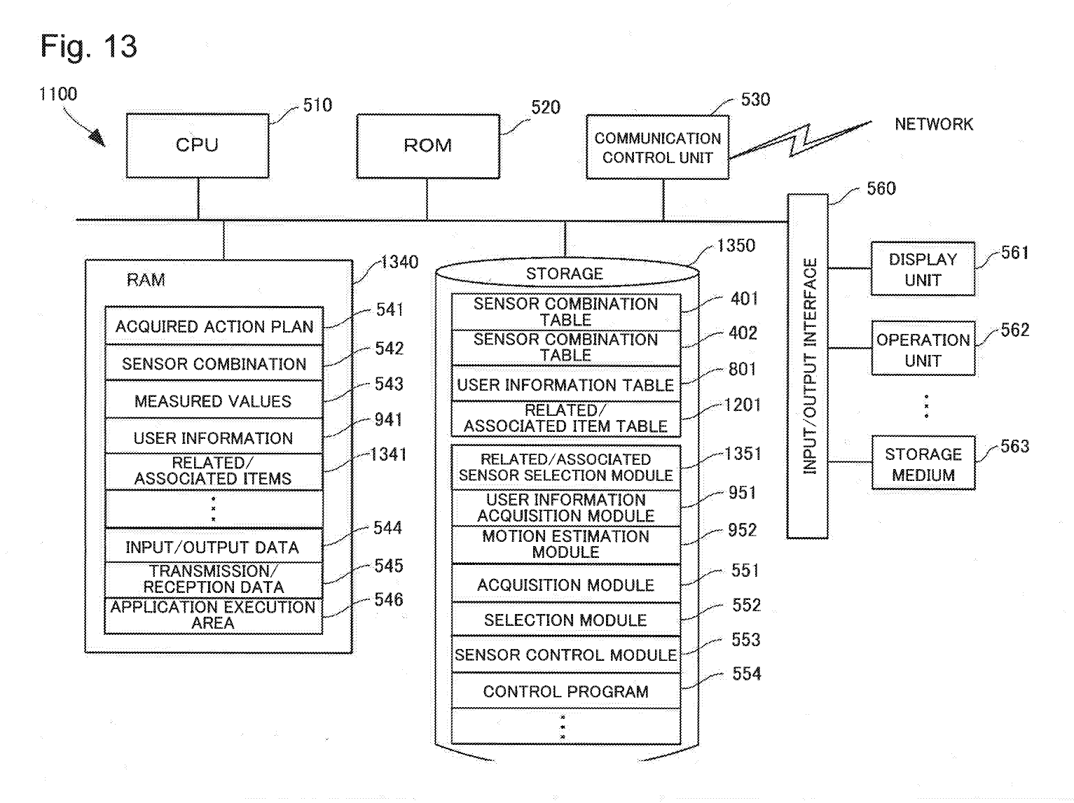

FIG. 13 is a block diagram illustrating a hardware configuration of the information processing device 1100 according to the present example embodiment. A RAM 1340 is a random access memory used by the CPU 510 as a temporary-storage work area. An area for storing data required for implementing the present example embodiment is reserved in the RAM 1340. The related/associated items 1341 are loaded from the related/associate item table 1201 stored in a storage 1350, for example.

The storage 1350 stores a database and various parameters, or data or programs, which will be described below, required for implementing the present example embodiment. The related/associated item table 1201 is a table configured as illustrated in FIG. 12.

The storage 1350 further stores a related/associated sensor selection module 1351. The related/associate sensor selection module 1351 is a module that selects a sensor related to or associated with an acquired action plan. The related/associated sensor selection module 1351 is loaded by the CPU 510 into an application execution area 547 in the RAM 940 and executed in the application execution area 547.

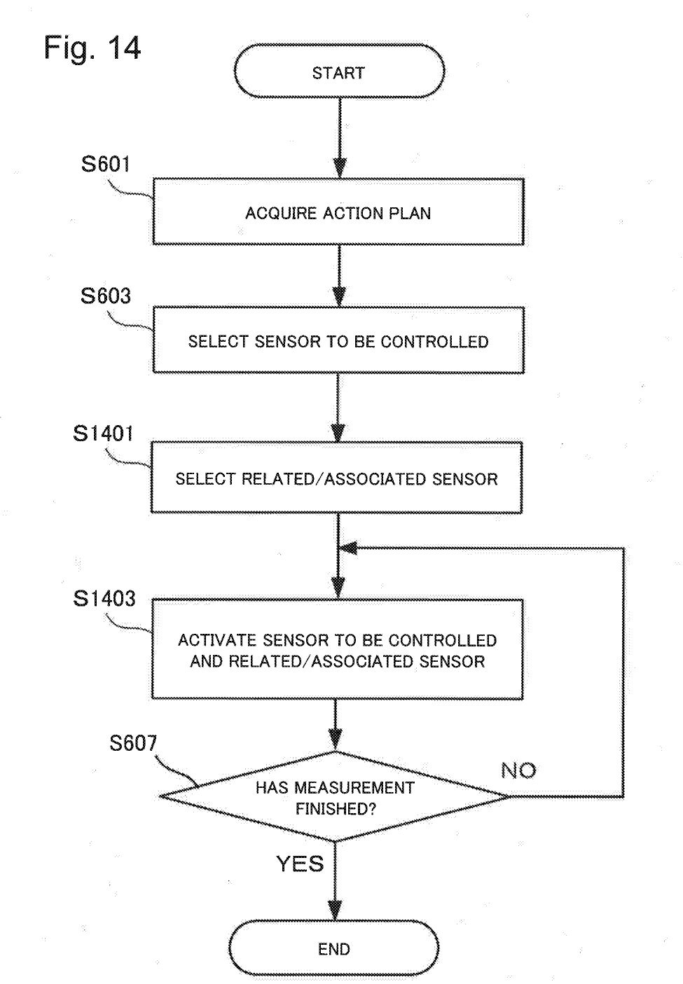

FIG. 14 is a flowchart illustrating a procedure of processing performed by the information processing device 1100 according to the present example embodiment. The flowchart is executed by the CPU 510 in FIG. 13 using the RAM 940 and implements the functional components of the information processing device 1100 in FIG. 11. In step S1401, the related/associate sensor selection 1101 selects a sensor 301 that is related to or associated with an action plan. In step S1403, the information processing device 1100 activates a sensor to be controlled and the related/associated sensor.

According to the present example embodiment, a combination of sensors can be selected in accordance with an action plan of a user and, in addition, a sensor associated with the selected sensors can be selected. Accordingly, the user can know predictive signals of an accident and injury due to an exercise, and disease, in addition to measuring his/her performance and the like and can prevent such an accident, injury and disease.

### OTHER EXAMPLE EMBODIMENTS

While the present invention has been described with reference to example embodiments, the present invention is not limited to the example embodiments described above. Various modifications that can be understood by those skilled in the art can be made to configurations and details of the present invention within the scope of the present invention.

Further, systems and devices that are any combinations of different features included in the example embodiments also fall within the scope of the present invention.

The present invention may be applied to a system made up of a plurality of devices or may be applied to a single device. Moreover, the present invention is also applicable to a case where an information processing program that implements functions of an example embodiment is provided directly or remotely to a system or a device. Accordingly, a program installed in a computer in order to implement functions of the present invention by the computer, or a medium that stores the program, and a World Wide Web (WWW) server that allows the program to be downloaded also fall within the scope of the present invention. In particular, a non-transitory computer readable medium that stores a program that causes a computer to execute processing steps included in at least the example embodiments described above falls within the scope of the present invention.

This application is based upon and claims the benefit of priority from the Japanese Patent Application No. 2015-186278 filed on Sep. 24, 2015, the entire disclosure of which is incorporated herein.

1. An information processing device configured to be attached to a body of a user, comprising:
a plurality of sensors;
an acquisition unit configured to acquire an action plan of the user; and
a control unit configured to select a sensor to be controlled among the plurality of sensors, based on the acquired action plan.

2. The information processing device according to claim 1, wherein
the control unit activates a sensor that is suitable for the action plan among the plurality of sensors.

3. The information processing device according to claim 1, wherein
the control unit deactivates a sensor that is not suitable for the action plan among the plurality of sensors.

4. The information processing device according to claim 1, further comprising a storage unit configured to store the action plan and the sensor to be controlled in association with each other.

5. The information processing device according to claim 1, further comprising a user information acquisition unit configured to acquire user information concerning the user, wherein
the control unit selects the sensor to be controlled, further based on the user information.

6. The information processing device according to claim 1, further comprising an estimation means that estimates a motion of the user, wherein
the control unit configured to select the sensor to be controlled, further based on a result of estimation by the estimation means.

7. The information processing device according to claim 1 further comprising a related/associated sensor selection unit configured to select a sensor related to or associated with the acquired action plan among the plurality of sensors.

8. The information processing device according to claim 1, wherein
the control unit controls at least one of sensitivity, a detection interval, and a detection frequency of the sensor.

9. The information processing device according to claim 1, wherein
the plurality of sensors include at least one of a heart rate sensor, an acceleration sensor, a GPS sensor, an ambient temperature sensor, a body temperature sensor, an altitude sensor, a humidity sensor, a lactate level sensor, a respiratory rate sensor, a blood flow sensor, an oxygen level sensor, a blood-sugar-level sensor, a perspiration sensor, and an alcohol sensor.

10. A control method for an information processing device that includes a plurality of sensors and is configured to be attached to a body of a user, the control method comprising:
acquiring an action plan of the user; and
selecting a sensor to be controlled among the plurality of sensors, based on the acquired action plan.

11. A non-transitory computer readable storage medium that stores control program for an information processing device that includes a plurality of sensors and is configured to be attached to a body of a user, the control program causing a computer to execute:
acquiring an action plan of the user; and
selecting a sensor to be controlled among the plurality of sensors, based on the acquired action plan.

12. The information processing device according to any one of claim 2, further comprising a storage unit configured to store the action plan and the sensor to be controlled in association with each other.

13. The information processing device according to any one of claim 3, further comprising a storage unit configured to store the action plan and the sensor to be controlled in association with each other.

14. The information processing device according to any one of claim 2, further comprising a user information acquisition unit configured to acquire user information concerning the user, wherein
the control unit selects the sensor to be controlled, further based on the user information.

15. The information processing device according to any one of claim 3, further comprising a user information acquisition unit configured to acquire user information concerning the user, wherein
the control unit selects the sensor to be controlled, further based on the user information.

16. The information processing device according to any one of claim 4, further comprising a user information acquisition unit configured to acquire user information concerning the user, wherein
the control unit selects the sensor to be controlled, further based on the user information.

17. The information processing device according to claim 2, further comprising an estimation means that estimates a motion of the user, wherein
the control unit configured to select the sensor to be controlled, further based on a result of estimation by the estimation means.

18. The information processing device according to claim 3, further comprising an estimation means that estimates a motion of the user, wherein
the control unit configured to select the sensor to be controlled, further based on a result of estimation by the estimation means.

19. The information processing device according to claim 4, further comprising an estimation means that estimates a motion of the user, wherein
the control unit configured to select the sensor to be controlled, further based on a result of estimation by the estimation means.

20. The information processing device according to claim 5, further comprising an estimation means that estimates a motion of the user, wherein
the control unit configured to select the sensor to be controlled, further based on a result of estimation by the estimation means.
